(19) United States
(12) Patent Application Publication
(10) Pub. No.: US 2026/0350786 A1
(43) Pub. Date: Nov. 5, 2026

(54) PHYSIOLOGICAL SIGNAL EXTRACTION FROM FACIAL VIDEO

(71) Applicant: West Virginia University Board of Governors on behalf of West Virginia University, Morgantown, WV (US)

(72) Inventors: Nima Karimian, Morgantown, WV (US); Banafsheh Adami, Morgantown, WV (US)

(21) Appl. No.: 19/182,758

(22) Filed: Apr. 18, 2025

(51) Int. Cl. G06T 7/00; A61B 5/00; A61B 5/024; A61B 5/327; G06T 5/60; G06T 5/70; G06V 10/25; G06V 10/77; G06V 10/82; G06V 20/40; G06V 40/10; G06V 40/16

(52) U.S. Cl. CPC G06T 7/0012; A61B 5/02416; A61B 5/327; A61B 5/7203; A61B 5/7267; G06T 5/60; G06T 5/70; G06V 10/7715; G06V 10/82; G06V 20/46; G06V 40/15; G06V 40/168; A61B 2576/02; G06T 2207/10016; G06T 2207/20036; G06T 2207/20081; G06T 2207/20084; G06T 2207/30088; G06T 2207/30201; G06V 10/25; G06V 2201/03

(57) ABSTRACT

Reconstruction of physiological signals from facial video sequences is discussed. One example method includes extracting a raw physiological signal from a facial video sequence. The method also includes encoding the raw physiological signal into a latent representation of the raw physiological signal. The method additionally includes generating a noisy latent representation by applying forward diffusion to progressively add noise to the latent representation over a first set of iterations. The method further includes generating a denoised latent representation by applying reverse diffusion to progressively remove noise from the noisy latent representation over a second set of iterations. Additionally, the method includes decoding the denoised latent representation to generate a reconstructed physiological signal.

### STATEMENT REGARDING FEDERALLY SPONSORED RESEARCH

This invention was made with government support under grant 2338981 awarded by the National Science Foundation. The government has certain rights in the invention.

### TECHNICAL FIELD

This description relates to reconstruction of physiological signals such as electrocardiogram (ECG) and/or photoplethysmography (PPG) signals from analysis of facial video.

### BACKGROUND

Cardiovascular disease is the leading cause of death in the world. However, early detection of cardiovascular disease can significantly improve treatment and outcomes. Continuous non-invasive cardiac monitoring can enable early detection and prevention of cardiovascular diseases. Traditional electrocardiogram (ECG) and photoplethysmography (PPG) sensors detect based on physical contact with the skin, limiting their usability in real-world, continuous monitoring scenarios. In addition to healthcare applications, the distinctive morphology of PPG and ECG signals has been successfully used as a biometric identifier, or it can be utilized as liveness detection for biometric anti-spoofing techniques.

### SUMMARY

A first example relates to a non-transitory machine-readable medium having machine executable instructions for a physiological signal reconstruction system that cause a processor core to execute operations. The operations include extracting a raw physiological signal from a facial video sequence. The operations also include encoding the raw physiological signal into a latent representation of the raw physiological signal. The operations additionally include generating a noisy latent representation by applying forward diffusion to progressively add noise to the latent representation over a first set of iterations. The operations further include generating a denoised latent representation by applying reverse diffusion to progressively remove noise from the noisy latent representation over a second set of iterations. Additionally, the operations include decoding the denoised latent representation to generate a reconstructed physiological signal.

A second example relates to a non-transitory machine-readable medium having machine executable instructions for training a physiological signal reconstruction system that cause a processor core to execute operations. The operations include accessing a training dataset comprising a set of training samples. A training sample of the set of training samples comprises a facial video sequence and a ground truth physiological signal synchronized with the facial video. The operations also include extracting a set of raw physiological signals from facial video sequences of the set of training samples. A raw physiological signal of the set of raw physiological signals is extracted from the facial video sequence via a three-dimensional convolutional neural network (3DCNN). The operations additionally include encoding the set of raw physiological signals into a set of latent representations. A latent representation of the set of latent representations is generated by encoding the raw physiological signal via an encoder network. The operations further include generating a set of noisy latent representations from the set of latent representations. A noisy latent representation of the set of noisy latent representations is generated via forward diffusion by progressively adding noise to the latent representation over a first set of iterations. Additionally, the operations include training a reverse diffusion model to generate a set of denoised latent representations from the set of noisy latent representations. A denoised latent representation of the set of denoised latent representations is generated by progressively removing noise from the noisy latent representation over a second set of iterations. The reverse diffusion model is trained based on the ground truth physiological signal and a reconstructed physiological signal generated by decoding the denoised latent representation.

A third example relates to a method. The method includes extracting a raw physiological signal from a facial video sequence. The method also includes encoding the raw physiological signal into a latent representation of the raw physiological signal. The method additionally includes generating a noisy latent representation by applying forward diffusion to progressively add noise to the latent representation over a first set of iterations. The method further includes generating a denoised latent representation by applying reverse diffusion to progressively remove noise from the noisy latent representation over a second set of iterations. Additionally, the method includes decoding the denoised latent representation to generate a reconstructed physiological signal.

### BRIEF DESCRIPTION OF THE DRAWINGS

FIG. 1 illustrates an example computing environment implementing a physiological signal reconstruction system capable of reconstructing a physiological signal (e.g., photoplethysmography (PPG) and/or electrocardiogram (ECG), etc.) from a facial video sequence.

FIG. 2 illustrates an example PPG waveform with multiple regions of interest (e.g., fiducial points) identified.

FIG. 3 illustrates an example ECG waveform with multiple regions of interest (e.g., fiducial points) identified.

FIG. 4 illustrates a flow diagram of a method for generating a reconstructed physiological signal based on a facial video sequence.

FIG. 5 illustrates a flow diagram of a method for training a diffusion model to generate reconstructed physiological signal(s) based on facial video sequence(s).

### DETAILED DESCRIPTION

Various example systems and methods described herein provide techniques for waveform reconstruction of remote PPG (rPPG) and/or remote ECG (rECG) from facial video. Examples employ a diffusion model (e.g., a Hamiltonian Monte Carlo diffusion model (HMCDM), for example, an entropy-regulated HMCDM (ER-HMCDM), etc.) to extract latent features determined from a facial video (e.g., from the output of a 3D CNN applied to facial video, etc.) and generate ECG and/or PPG signals. In some examples, reconstructed signals are used for biometrics, health monitoring, stress monitoring, etc.

Additionally, existing databases for training models lack both ECG and PPG signals, provide only heart rate values from PPG with ECG waveforms, and/or are limited in terms of subject diversity and skin tone representation. As such, in various examples, training of models discussed herein is based at least in part on a database of facial video recordings (e.g., around 13 hours) synchronized with high-resolution ECG and PPG signals from a broad range of genders, ethnicities, and skin tones.

Continuous non-invasive cardiac monitoring can provide multiple healthcare benefits such as early detection of cardiac conditions, while overcoming the limitations of traditional methods of electrocardiograms (ECG) and photoplethysmography (PPG) that involve invasive electrodes and constant supervision. Using both PPG and ECG together can provide a more comprehensive understanding of cardiovascular health, as PPG and ECG offer complementary information on heart rate, rhythm, and blood flow.

Various examples employ diffusion model(s) for reconstruction of PPG and/or ECG signals, including HMCDM(s) and/or ER-HMCDM(s). Unlike existing denoising diffusion probabilistic models (DDPMs), various examples incorporate Hamiltonian Monte Carlo sampling into the reverse diffusion process, which in some examples is combined with entropy regularization. By maximizing entropy, examples employing entropy regularization promote diversity in the generated waveforms, effectively exploring the latent space and mitigating the risk of mode collapse. Additionally, various examples employ region-of-interest (ROI)-guided diffusion techniques that integrate guided attention mechanisms into the diffusion process, enabling the model to prioritize physiologically significant regions of the signal, such as fiducial points and/or selected waveform transitions. Examples employing ROI-guided diffusion techniques ensure the accurate reconstruction of fine-grained morphological details, such as systolic peaks and diastolic notches from the PPG signal, and the P, QRS complex, and T waveform from the ECG signal, which are features useable for detecting heart disease. Accordingly, various examples accurately reconstruct PPG and/or ECG signals based on facial video sequences, providing more detailed information beyond beats-per-minute (BPM) or heart rate (HR) estimation.

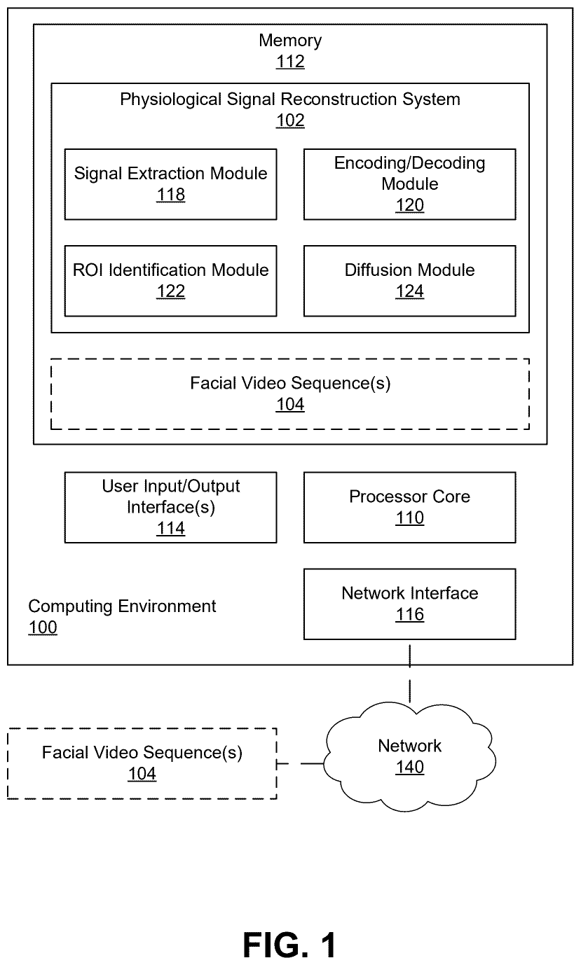

FIG. 1 illustrates an example computing environment 100 implementing a physiological signal reconstruction system 102 capable of generating a reconstructed physiological signal (e.g., ECG, PPG, etc.) based on an input facial video sequence. The physiological signal reconstruction system 102 extracts latent features from a facial video sequence of facial video sequence(s) 104 and applies a diffusion model to generate the reconstructed physiological signal. In various examples, the facial video sequence(s) 104 are stored locally at the computing environment 100 (e.g., as shown within the memory 112, etc.) and/or remotely (e.g., as shown connected to the computing environment 100 via the network 140).

The computing environment 100 includes a processor core 110, a memory 112, a user input/output (I/O) interface 114, and a network interface 116, which are operably connected for computer communication. The processor core 110 performs general computing to execute instructions stored in the memory 112, including instructions associated with the physiological signal reconstruction system 102. The instructions cause the processor core 110 to execute operations. The memory 112 also stores instructions associated with an operating system that controls and/or allocates resources of computing environment 100, including resources associated with the physiological signal reconstruction system 102. Memory 112 represents a non-transitory machine-readable memory (or other medium), such as random access memory (RAM), a solid state drive, a hard disk drive or a combination thereof.

Processor core 110 accesses memory 112 and executes the machine-readable instructions as operations. Processor core 110 can be a variety of various processors including multiple single- and multi-core processors, co-processors, and other multiple single and multicore processor and co-processor architectures.

User I/O interface 114 provides software and hardware to facilitate data input and output between computing environment 100 and a user. This can include input devices such as a keyboard, mouse, touchpad, touchscreen, microphone, camera, etc., as well as output devices such as display(s) (e.g., light-emitting diode (LED) display panel(s), liquid crystal display (LCD) panel(s), plasma display panel(s), and/or touch screen display(s), etc.), speaker(s), etc. User I/O interface 114 provides graphical input controls for a user interface, which can include software and hardware-based controls, interfaces, touch screens, or touch pads or plug and play devices for a user to provide user input.

The memory 112 includes the physiological signal reconstruction system 102, which includes a signal extraction module 118, an encoding/decoding module 120, a region-of-interest (ROI) identification module 122, and a diffusion module 124 that operate in concert and/or stages to reconstruct a physiological signal (e.g., a PPG signal, an ECG signal, etc.) based on a facial video sequence. In various examples, the physiological signal reconstruction system 102 extracts a raw physiological signal from the facial video sequence 104 (e.g., via a three-dimensional convolutional neural network (3DCNN), etc.) that is encoded into a latent representation. The physiological signal reconstruction system 102 of various examples uses forward and reverse diffusion (e.g., via a Hamiltonian Monte Carlo (HMC) diffusion model (HMCDM), for example, with entropy regularization and/or via a ROI-guided process, etc.) to generate a denoised latent representation. Various examples of the physiological signal reconstruction system 102 decode the denoised latent representation into the reconstructed physiological signal (e.g., an ECG signal, a PPG signal, etc.).

The signal extraction module 118 extracts a raw physiological signal from the facial video sequence 104. In various examples, the signal extraction module 118 employs a three-dimensional convolutional neural network (3DCNN) to extract the raw physiological signal. Given a facial video sequence

$$V = {\{ {V}_{t} \}}_{t = 1}^{T} ,$$

where each frame Vₜ∈ [symbol] ^{H×W×3} represents an RGB image, the signal extraction module 118 extracts spatial and temporal features using the 3DCNN as in equation (1):

X_{signal}=f_{3DCNN}(V) (1)

where f_{3DCNN} represents the 3DCNN extracting the raw physiological signal X_{signal} from facial video frames.

In various examples, to enhance feature representation and facilitate structured signal reconstruction, the encoding/decoding module 120 encodes the raw physiological signal X_{signal} into a latent representation Z₀ as in equation (2):

Z₀=f_{enc}(X_{signal}) (2)

where in various examples f_{enc} is an encoder network constructed from multiple CNN layers that transforms X_{signal} into a compact latent representation Z₀. The latent representation Z₀ retains relevant temporal and morphological characteristics while filtering out noise and redundant variations.

In various examples, to guide the diffusion process, the ROI identification module 122 identifies ROIs from the extracted and/or encoded signal, such as fiducial points I_{fiducial} corresponding to relevant physiological landmarks, such as systolic peaks and diastolic notches (for PPG) or PQRST complexes (for ECG). In various examples, these ROIs (e.g., fiducial points, etc.) are used in the noise perturbation and reverse denoising stages to ensure that various signal characteristics remain preserved throughout the forward and reverse diffusion process.

In various examples, the diffusion module 124 performs forward diffusion (e.g., noise perturbation, etc.) on the latent representation to generate a noisy latent representation. The diffusion module 124 in various examples applies reverse diffusion to the noisy latent representation to generate a denoised latent representation \hat{Z}₀. In some examples, the diffusion module 124 employs a Hamiltonian Monte Carlo Diffusion Model (HMCDM) for reverse diffusion. Additionally, the diffusion module 124 in some examples applies ROI-aware forward and reverse diffusion and/or employs entropy regularization to curtail mode collapse and enhance feature preservation.

After the reverse diffusion process, the denoised latent representation \hat{Z}₀ is mapped back to the signal space by the encoding/decoding module 120 using a decoder as in equation (3):

Ŷ_{PPG/ECG}=f_{dec}(\hat{Z}₀) (3)

where in various examples f_{dec} is a decoder network (e.g., constructed from a transpose CNN, etc.) that reconstructs the final physiological signal (e.g., PPG or ECG waveform, etc.) from the denoised latent representation \hat{Z}₀ and Ŷ_{PPG/ECG} represents the output physiological (e.g., PPG, ECG, etc.) signal after reconstruction. The encoding/decoding module 120 employs the decoder to ensure that the reconstructed physiological signal maintains physiological fidelity while mitigating noise and artifacts introduced during the diffusion process.

In various examples, forward diffusion is applied (e.g., by the diffusion module 124, etc.) in a ROI-aware manner. In the forward diffusion process, noise is progressively added to the latent representation Z over T timesteps. In some examples, instead of applying the same noise to the entire latent representation, a ROI constraint is employed to ensure that ROIs such as identified physiological structures (e.g., fiducial points, etc.) are less affected by noise. In some such examples, the forward diffusion process is applied according to equation (4):

$${Z}_{t} [ i ] = \{ \begin{matrix}\sqrt{{\overline{α}}_{t}} {Z}_{0} [ i ] + \sqrt{( 1 - {\overline{α}}_{t} ) \cdot γ} {ϵ}_{t} [ i ] , & i ∈ {I}_{fiducial} \\ \sqrt{{\overline{α}}_{t}} {Z}_{0} [ i ] + \sqrt{( 1 - {\overline{α}}_{t} )} {ϵ}_{t} [ i ] , & otherwise\end{matrix} \qquad ( 4 )$$

where Zₜ[i] is the noisy latent representation at timestep t, Z₀[i] is the original clean latent representation before diffusion, at is the noise schedule that controls the noise addition at timestep t, ϵₜ[i]~ [symbol] (0, I) is Gaussian noise sampled independently at each timestep, I_{fiducial} is the set of indices corresponding to fiducial points (e.g., the identified ROI(s), such as systolic peaks or diastolic notches for PPG, PQRST complexes for ECG, etc.). The forward diffusion model preserves physiological features by scaling using γ at fiducial indices, such that selected waveform features (e.g., the ROI(s), etc.) remain intact, improving reconstruction accuracy. Standard diffusion noise is applied to regions outside the ROI(s) (e.g., non-fiducial regions, etc.), allowing stochastic variations while mitigating degradation of identified signal structures.

The difference of forward ROI constraints with the standard diffusion process without ROI constraints (e.g., which is applied in some examples) is given by Zₜ=\sqrt{āₜ}Z₀+\sqrt{(1−āₜ)}ϵₜ, where noise is applied uniformly across all indices. In contrast, examples employing ROI-guided diffusion selectively reduce noise at the ROI(s) (e.g., fiducial locations, etc.), facilitating high-fidelity waveform reconstruction.

In various examples, the reverse diffusion that is applied (e.g., by the diffusion module 124, etc.) leverages Hamiltonian dynamics to sample from the posterior distribution, ensuring structured and efficient denoising. The Hamiltonian function employed in various examples is shown in equation (5):

$$H ( Z , ω ) = U ( Z ) + V ( ω ) - λ {\sum }_{i ∈ {I}_{fiducial}} {H}_{entropy} ( Z [ i ] ) \qquad ( 5 )$$

where H(Z, ω) is the Hamiltonian function, U(Z)=−log π(Z) is the potential energy enforcing physiological consistency,

$$V ( ω ) = \frac{1}{2} {‖ ω ‖}^{2}$$

represents the kinetic energy term, H_{entropy}(Z)=−ΣZ log(Z+ϵ) is the entropy regularization function, and λ is a hyperparameter controlling the trade-off between entropy regularization and structure preservation (for examples applying entropy regularization, λ is nonzero).

To iteratively refine the latent representation, various examples employ numerical integration, such as the Leapfrog integration of equations (6)-(8). Equation (6) shows the momentum update for the first half-step:

$${ω}_{t + \frac{1}{2}} = {ω}_{t} - \frac{ϵ}{2} {∇}_{Z} U ( {Z}_{t} ) + \frac{ϵ}{2} λ {\sum }_{i ∈ {I}_{fiducial}} {∇}_{Z} {H}_{entropy} ( Z [ i ] ) \qquad ( 6 )$$

Equation (7) shows the latent state update:

$${Z}_{t + 1} = {Z}_{t} + {ϵω}_{t + \frac{1}{2}} \qquad ( 7 )$$

Equation (8) shows the momentum update for the final half-step:

$${ω}_{t + 1} = {ω}_{t + \frac{1}{2}} - \frac{ϵ}{2} {∇}_{Z} U ( {Z}_{t + 1} ) + \frac{ϵ}{2} λ {\sum }_{i ∈ {I}_{fiducial}} {∇}_{Z} {H}_{e n t r o p y} ( Z [ i ] ) \qquad ( 8 )$$

where ωₜ is the momentum variable at time t, Zₜ represents the latent variable at time t, ϵ is the Leapfrog step size, ∇_{Z}U(Zₜ) computes the gradient of potential energy with respect to Zₜ, and λ∇_{Z}H_{entropy}(Zₜ) applies entropy regularization, mitigating mode collapse and enhancing feature preservation. In one example, λ=0.5, although greater or lesser values are used in some examples.

In various examples, to ensure the preservation of identified physiological structures, a ROI mask is applied (e.g., by the diffusion module 124 based on ROIs identified by the ROI identification module 122, etc.) throughout both the forward and reverse diffusion processes.

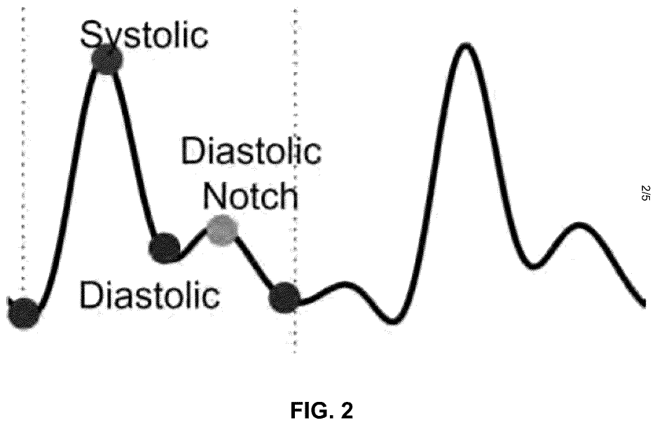

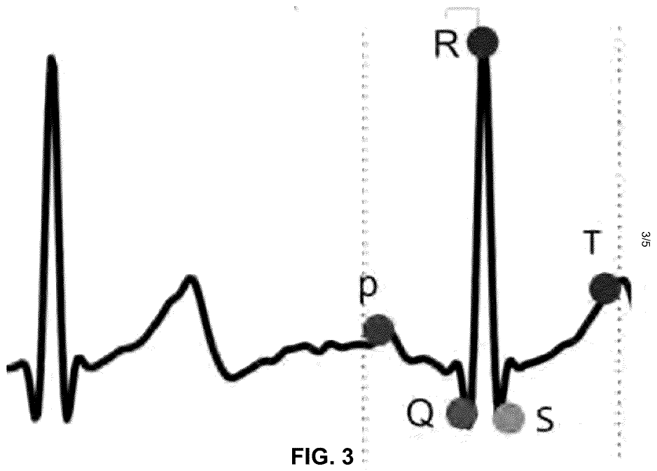

For ROI-guided diffusion, ROIs (e.g., fiducial regions, etc.) are detected (e.g., by the ROI identification module 122, etc.) within the signal (e.g., the raw physiological signal, etc.) to determine identified waveform landmarks. The ROIs identified in different physiological waveforms (e.g., the PPG and ECG waveforms, etc.) vary based on the specific physiological waveform and can also vary between examples. Referring to FIG. 2, illustrated is an example PPG waveform with multiple ROIs (e.g., fiducial points) identified. The example ROIs identified in the PPG waveform of FIG. 2 include a systolic peak (e.g., X_{systolic}, the highest peak in the PPG cycle, corresponding to the maximum arterial blood volume), a diastolic notch (e.g., X_{notch}, a secondary inflection point occurring due to vascular elasticity), and a diastolic peak (e.g., X_{diastolic}, the lowest point in the cycle before the next systolic upstroke). Referring to FIG. 3, illustrated is an example ECG waveform with multiple ROIs (e.g., fiducial points) identified. The example ROIs identified in the ECG waveform of FIG. 3 include a P-wave (e.g., X_{P}, representing atrial depolarization), a QRS complex (e.g., X_{QRS}, a rapid succession of changes indicating ventricular depolarization), and a T-wave (e.g., X_{T}, representing ventricular repolarization).

To enhance the ability to reconstruct high-fidelity waveforms, various examples integrate ROI-specific constraints into the entropy regularization term during Hamiltonian Monte Carlo diffusion. In such examples, the entropy function is reformulated as in equation (9):

$${H}_{entropy} ( X ) = - {\sum }_{i} {X}_{i} log ( {X}_{i} + ϵ ) \qquad ( 9 )$$

where the summation is performed over all ROI(s) selected (e.g., ROI-selected fiducial points such as X_{systolic}, X_{notch}, and X_{diastolic} for PPG; X_{P}, X_{QRS}, and X_{T} for ECG; etc.). By selectively incorporating entropy constraints on selected ROIs (e.g., fiducial points, etc.), such examples ensure that the diffusion process prioritizes the most physiologically significant waveform structures.

Algorithm (1) provides pseudocode for one example using ROI-guided and entropy-regularized Hamiltonian Monte Carlo Diffusion:

Algorithm 1: ROI-Aware Entropy-Regularized Hamiltonian Monte Carlo
Diffusion (ER-HMCDM)

```
  1:  $Input : Facial video sequence V = {\{ {V}_{t} \}}_{t = 1}^{T}$
  2: Output: Reconstructed PPG and/or ECG waveform Ŷ_{PPG/ECG}
  3: Step 1: Extract Physiological Signal
  4: Extract raw physiological signal from 3DCNN
  5: Encode extracted signal into latent representation.
  6: Extract fiducial points.
  7: Step 2: Forward Diffusion Process (ROI-Aware)
  8: for each timestep t from 1 to T do
  9: Apply ROI-aware noise perturbation.
10: end for
11: Step 3: Reverse Diffusion with ROI-Constrained ER-HMCDM
12: for each timestep t from T to 1 do
13: Compute Hamiltonian function.
14: Perform Leapfrog integration:
15: Momentum update (first half-step)
16: Latent state update
17: Momentum update (final half-step)
18: Apply ROI correction.
19: end for
20: Step 4: Decode and Output Reconstructed Signal
21: Decode from latent space and output the reconstructed signal.
```

In various examples, diffusion models (e.g., employed by the diffusion module 124, etc.) are trained on one or more datasets that include a training set of facial video sequences (e.g., from the facial video sequence(s) 104, etc.) along with PPG (e.g., from leads or rPPG) and/or ECG (e.g., from leads or rECG) signals that are synchronized with the facial video sequences of the training set. While numerous datasets exist for rPPG-based heart rate estimation, many do not include actual PPG signals. Instead, they provide only heart rate values or blood pressure, which differ from PPG waveforms.

Some examples are trained at least in part on a dataset (e.g., a PhysioFace Diverse Dataset (PFDD), etc.) created in connection with various examples to reflect the diversity and complexity of real-world video-based physiological monitoring applications. One hundred participants were selected to represent a range of ages, genders, ethnicities, facial structures and skin tones (e.g., including multiple participants representing each Fitzgerald skin type). Each participant was seated in a controlled environment to minimize motion artifacts and was equipped with a Shimmer3 GSR+ unit featuring an optical pulse sensor attached to the index finger to record PPG signals. Additionally, participants wore a Vernier EKG sensor with three electrodes placed on the left arm and both right arms. A total of 480 seconds of video footage was recorded per participant using a Nikon D700 DSLR camera, while ground-truth PPG and ECG signals were simultaneously captured using the Shimmer3 GSR+ and Vernier EKG sensors, respectively. The PPG and ECG signals were sampled at 125 Hz, with each recording session lasting eight minutes. The video recordings were captured at 24 frames per second with a resolution of 1920×1080 pixels. To synchronize the signals with the video frame rate, the PPG and ECG signals were downsampled from 125 Hz to 24 Hz, yielding 1440 frames per minute of video. The dataset includes multiple demographic labels such as skin tone, gender, ethnicity, hair coverage, makeup, and presence of eyeglasses to support robust physiological signal reconstruction across a range of demographic-related factors.

To facilitate accurate reconstruction of ECG and/or PPG signals, various examples employ a supervised process to train the diffusion model (e.g., via the diffusion module 124). In some examples, the reverse diffusion and/or supervised process employs a multi-component loss function, such as in equation (10):

[symbol]

=λ₁ [symbol] _{NP}+λ₂ [symbol] _{RG}+λ₃ [symbol] _{fid} (10)

where: [symbol] _{NP} is the noise prediction loss, which minimizes the discrepancy between the predicted and actual noise during diffusion; [symbol] _{RG} is the region-guided refinement loss, which enhances selected physiological regions (e.g., for PPG, [symbol] _{RG} refines systolic peaks, diastolic notches, and diastolic peaks; for ECG, [symbol] _{RG} prioritizes PQRST complexes for accurate cardiac morphology; etc.); and [symbol] _{fia} is the fiducial point loss, which ensures waveform peak alignment between reconstructed and ground truth signals. In various examples, loss(es) included in equation (10) are able to be used in training the diffusion model of that example by setting the associated weighting hyperparameter (λ₁, λ₂, and/or λ₃) to a nonzero value. In some examples, the weighting hyperparameters are set such that λ₁ is greater than λ₂ and λ₃. One example has values of λ₁=0.5, λ₂=0.3 and λ₃=0.2 to balance different loss components, although some other examples use different values for one or more of λ₁, λ₂, and/or λ₃. The combination of losses in equation (10) facilitates a robust, noise-invariant, and physiologically accurate reconstruction.

The noise prediction loss minimizes the difference between the predicted noise and the actual noise introduced during the forward diffusion process, and in various examples is formulated as in equation (11):

$${ℒ}_{N P} = {𝔼}_{t , {X}_{t} , ϵ} [ {‖ {ϵ}_{t} - {\hat{ϵ}}_{t} ‖}_{2}^{2} ] \qquad ( 11 )$$

where ϵₜ represents the actual noise added to the signal at time step t and \hat{ϵ}ₜ is the predicted noise output from the model. The expectation is taken over all time steps t and input signal instances Xₜ.

To improve the reconstruction of key physiological regions, the region-guided refinement (RG) loss applies stronger constraints on ROI(s) (e.g., fiducial points) and waveform morphology. The RG loss is computed as in equation (12):

$${ℒ}_{R G} = {\sum }_{i ∈ {I}_{fiducial}} ( {ω}_{i} \cdot \vert {X}_{r e c o n} ( i ) - {X}_{g t} ( i ) \vert ) \qquad ( 12 )$$

where I_{fiducial} is the set of identified ROIs (e.g., fiducial points such as systolic peaks, diastolic notches/peaks for PPG; PQRST complexes for ECG; etc.), ω₈ is a weighting factor that assigns higher importance to the ROIs, and X_{recon}(i) and X_{gt}(i) are the reconstructed and ground-truth signals at index i, respectively.

The fiducial point loss ensures accurate alignment of waveform peaks and ROIs (e.g., fiducial points, etc.) between the reconstructed and ground-truth signals, and is defined as in equation (13):

$${ℒ}_{f i d} = {\sum }_{i ∈ {I}_{fiducial}} {‖ {X}_{recon} ( i ) - {X}_{g t} ( i ) ‖}_{2}^{2} \qquad ( 13 )$$

The fiducial point loss penalizes misalignment of identified waveform ROI(s) (e.g., fiducial points, etc.), improving both time-domain and frequency-domain fidelity.

Equations (10)-(13) define a multi-component loss function used in various examples to facilitate accurate and physiologically consistent signal reconstruction. The guided loss of equation (12) and the fiducial loss of equation (13) are used in various examples to enhance the structural fidelity of the reconstructed physiological waveforms (e.g., PPG, ECG, etc.).

The guided loss is designed to direct the diffusion model's attention to physiologically meaningful regions of the PPG waveform (e.g., systolic peaks and diastolic notches/peaks) and/or ECG waveform (e.g., P-wave, QRS complex, and T-wave). These features facilitate derivation of clinically relevant parameters such as heart rate variability (HRV) and pulse transit time (PTT). By enforcing a stronger focus on these key regions, guided loss reduces distortions and improves peak alignment.

The fiducial loss ensures that the generated waveform maintains precise timing and amplitude relationships between identified fiducial points. Small shifts in these points can lead to substantial errors in cardiovascular assessments, thus accurate reconstruction substantially improves assessments based on the reconstructed physiological signal(s). The fiducial loss function penalizes deviations from the expected locations of ROIs (e.g., systolic peaks, diastolic troughs, and other identified landmarks), preserving the overall structure of the signal.

The inclusion of guided loss and/or fiducial loss improves signal reconstruction quality. Examples employing guided loss improve peak alignment, reducing errors in amplitude estimation, while examples employing fiducial loss reduce distortions at identified ROIs (e.g., fiducial points, etc.), improving the reliability of clinical parameter extraction. Examples that incorporate both losses achieve more accurate and clinically relevant waveform reconstructions, improving the suitability of the reconstructed physiological signals for a range of applications, such as cardiovascular monitoring, biometric signal processing, etc.

One specific example was implemented in PyTorch and trained on 2×NVIDIA RTX A5000 GPUs with 32 GB of VRAM. The AdamW optimizer was used with a learning rate of 10⁻⁴. The noise schedule parameters (β₁, β₂) 360 were set to (1×10⁴, 0.2), and the total diffusion timesteps (T) were set to 10. Entropy regularization was applied with λ=0.5. Training was conducted over 10000 epochs, and results were averaged across multiple runs for reproducibility. The model utilized an attention-based architecture with 8 attention heads in the cross-attention layers. Each ECG and PPG signal of the training set was segmented into 3-second windows, normalized, and upsampled to match the model's temporal resolution.

Various examples generate reconstructed physiological signals with sufficiently high fidelity for use in a variety of applications. These applications include non-contact cardiac monitoring, biometric authentication, cardiovascular disease detection, fatigue and/or stress detection, heart rate and oxygen saturation monitoring or variability tracking, biometric assessment for pre-screening and post-surgical monitoring, real-time physiological feedback, etc. In terms of biometric authentication, while either PPG signals or ECH signals are useable, ECG signals provide greater feature separation between subjects and easier authentication than PPG signals (as does the combination of ECG signal and PPG signal).

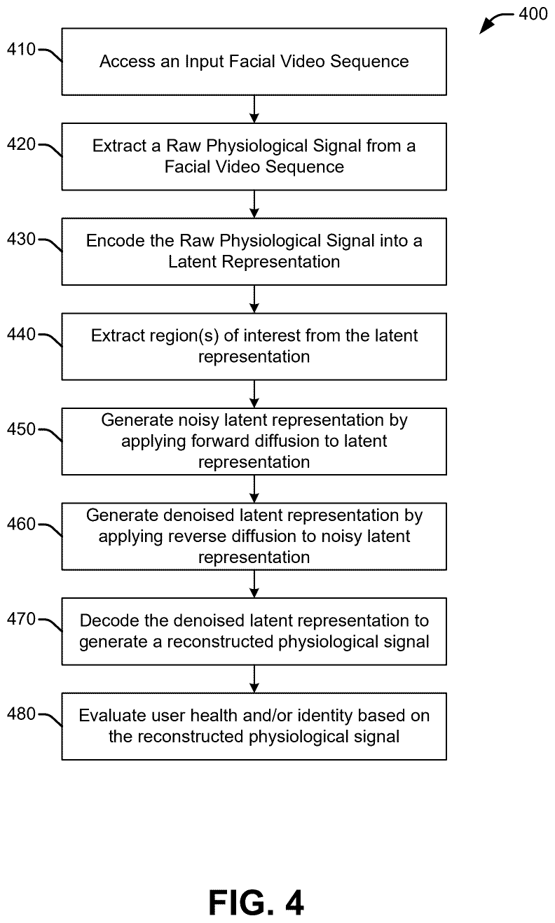

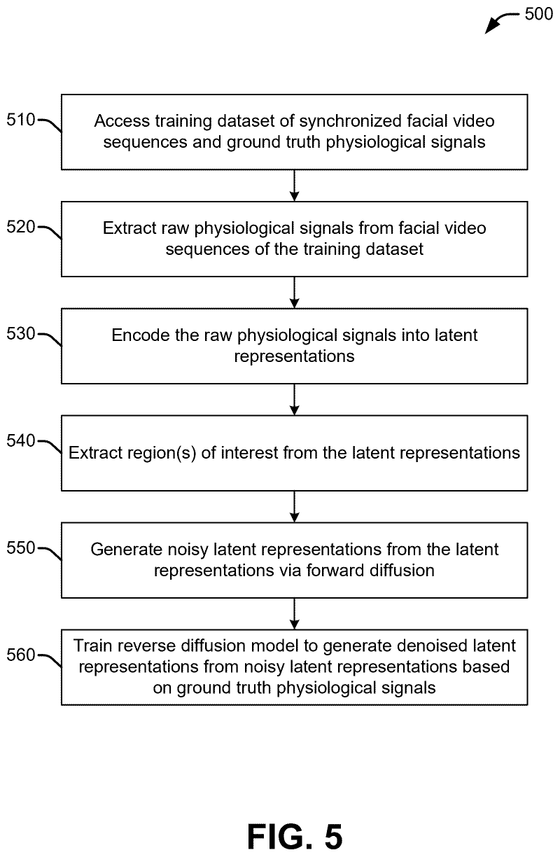

In view of the foregoing structural and functional features described above, example methods will be better appreciated with reference to FIGS. 4-5. While, for purposes of simplicity of explanation, the example methods of FIGS. 4-5 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method.

Referring to FIG. 4, illustrated is a flow diagram of a method 400 for generating a reconstructed physiological signal (e.g., PPG signal, ECG signal, etc.) based on a facial video sequence. In other examples, the blocks of the example method 400 are a set of machine-readable instructions on a non-transitory machine-readable medium or are a set of operations performed by a processor executing machine-readable instructions as the operations.

At block 410, method 400 includes accessing (e.g., via the computing device 100, etc.) an input facial video sequence (e.g.,

$$V = {\{ {V}_{t} \}}_{t = 1}^{T} ,$$

from the racial video sequence(s) 104, etc.).

At block 420, method 400 includes extracting (e.g., via the signal extraction module 118, etc.) a raw physiological signal (e.g., X_{signal}, etc.) from the facial video sequence. In various examples, a 3DCNN is employed to extract the raw physiological signal from the facial video sequence.

At block 430, method 400 includes encoding (e.g., via the encoding/decoding module 120, etc.) the raw physiological signal into a latent representation (e.g., Z₀, etc.) of the raw physiological signal.

At block 440, method 400 includes extracting (e.g., via the ROI identification module 122, etc.) one or more regions of interest (e.g., fiducial points I_{fiducial}, etc.) from the latent representation and/or the raw physiological signal. The ROIs extracted can vary between examples and based on the type of physiological signal. In various examples, ROIs for a PPG signal include at least one of a systolic peak, a diastolic notch, or a diastolic peak, while ROIs for an ECG signal include at least one of a P-wave, a QRS complex, or a T-wave.

At block 450, method 400 includes generating (e.g., via the diffusion module 124, etc.) a noisy latent representation (e.g., Zr, etc.) by progressively adding noise (e.g., per equation (4), etc.) to the latent representation over a first set of iterations (e.g., over T timesteps, etc.). In some examples, a first noise is progressively added to the one or more identified ROIs and a second noise (e.g., having a greater magnitude than the first noise, etc.) is progressively added to portions of the latent representation outside of the one or more ROIs.

At block 460, method 400 includes generating (e.g., via the diffusion module 124, etc.) a denoised latent representation (e.g., \hat{Z}₀, etc.) by applying reverse diffusion to progressively remove noise (e.g., according to equations (5)-(9), etc.) from the noisy latent representation over a second set of iterations (e.g., over T timesteps, etc.).

In various examples, applying the reverse diffusion includes numerically integrating (e.g., via Leapfrog integration, etc.) the noisy latent representation based on a Hamiltonian function (e.g., per equation (5), etc.) over the second set of iterations. In the same or other examples, the reverse diffusion employs entropy regularization, such as via including an entropy regularization function associated with the one or more ROIs in the Hamiltonian.

Additionally, in various examples, the reverse diffusion employs a multi-component loss function that is a weighted sum of a set of loss components (e.g., per equation (10), etc.). In such examples, the loss components include one or more of: a noise prediction loss (e.g., [symbol] _{NP}) that minimizes the discrepancy between a predicted noise and the noise added during forward diffusion, a region-guided refinement loss (e.g., [symbol] _{RG}) that applies additional constraints associated with the one or more regions of interest and a morphology of the reconstructed physiological signal, and/or a fiducial point loss (e.g., [symbol] _{fid}) that penalizes misalignment of the one or more ROIs.

At block 470, method 400 includes decoding (e.g., via the encoding/decoding module 120, etc.) the denoised latent representation to generate a reconstructed physiological signal (e.g., Ý_{PPG/ECG}, etc.).

At block 480, method 400 includes evaluating (e.g., via the computing environment 100, another computer, etc.) the health and/or identity of a user associated with the facial video sequence based on the reconstructed physiological signal. In some examples, the reconstructed physiological signal is analyzed by comparison with one or more previously stored (e.g., ground-truth, etc.) ECG and/or PPG signals associated with the user and/or one or more other users. As additional examples, the reconstructed physiological signal is analyzed to determine whether the reconstructed physiological signal is associated with a given health condition (e.g., a cardiovascular condition, stress, fatigue, etc.). In the same or other examples, the reconstructed physiological signal is analyzed to determine one or more characteristics of the reconstructed physiological signal, such as a heart rate, an oxygen saturation, etc.

In some examples, method 400 is performed a single time for the facial video sequence, and the reconstructed physiological sequence is a PPG signal or an ECG signal. In other examples, the method 400 is performed a first time on the facial video sequence for one of a PPG signal or an ECG signal and is also performed a second time on the facial video sequence for the other of the PPG signal or the ECG signal.

Referring to FIG. 5, illustrated is a flow diagram of a method 500 for training a diffusion model to generate reconstructed physiological signal(s) (e.g., PPG signal(s), ECG signal(s), etc.) based on facial video sequence(s). In other examples, the blocks of the example method 500 are a set of machine-readable instructions on a non-transitory machine-readable medium or are a set of operations performed by a processor executing machine-readable instructions as the operations.

At block 510, method 500 includes accessing (e.g., via the computing device 100, etc.) a training dataset (e.g., the PFDD dataset, etc.) that includes a set of training samples (e.g., as facial video sequence(s) 104, etc.). Each of the training samples includes a facial video sequence and at least one ground truth physiological signal (e.g., PPG and/or ECG, etc.) synchronized with the facial video sequence.

At block 520, method 500 includes extracting (e.g., via the signal extraction module 118, etc.) a set of raw physiological signals from the set of facial video sequences. In various examples, each raw physiological signal is extracted from an associated facial video sequence via a 3DCNN.

At block 530, method 500 includes encoding (e.g., via the encoding/decoding module 120, etc.) the set of raw physiological signals into a set of latent representations. In various examples, each raw physiological signal is encoded into an associated latent representation via an encoder network (e.g., constructed of multiple CNN layers, etc.).

At block 540, method 500 includes extracting (e.g., via the ROI identification module 122, etc.) a set of ROIs from the set of latent representations. In various examples, one or more ROIs are extracted from each latent representation of the set of latent representations.

At block 550, method 500 includes generating (e.g., via the diffusion module 124, etc.) a set of noisy latent representations from the set of latent representations via forward diffusion. In various examples, a noisy latent representation is generated from a latent representation by progressively adding noise (e.g., ROI-aware noise, etc.) to the latent representation over a first set of iterations.

At block 560, method 500 includes training (e.g., via the diffusion module 124, etc.) a reverse diffusion model (e.g., an ER-HMCDM, etc.) to generate a set of denoised latent representations from the set of latent representations. In various examples, a denoised latent representation is generated by progressively removing noise from an associated noisy latent representation over a second set of iterations. Additionally, in various examples, the reverse diffusion model is trained based on the ground truth physiological signal and a reconstructed signal generated by decoding (e.g., via the encoding/decoding module 120, etc.) the associated denoised latent representation.

In some examples, method 500 is performed a single time for a training set of facial video sequences, and the resulting model is trained to generate a reconstructed physiological sequence that is a PPG signal or an ECG signal. In other examples, the method 500 is performed a first time on the training set of facial video sequences to train a model to reconstruct one of PPG signal(s) or ECG signal(s) and is also performed a second time on the training set of facial video sequences to train a model to reconstruct other of PPG signal(s) or ECG signal(s).

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Also as used herein, the term “set” means one or more elements (e.g., where the elements can be anything, such as datasets, nodes, relationships, etc.), and a “subset” of a set A refers to any set B where every element of set B is an element of set A (note that every set A is a subset of itself, as every element of set A is an element of set A). Additionally, where the disclosure or claims recite “a,” “an,” “a first,” or “another” element, or the equivalent thereof, it should be interpreted to include one or more than one such element, neither requiring nor excluding two or more such elements.

In this description, unless otherwise stated, “about,” “approximately” or “substantially” preceding a parameter means being within +/−10 percent of that parameter. Modifications are possible in the described embodiments, and other embodiments are possible, within the scope of the claims.

What is claimed is:

1. A non-transitory machine-readable medium having machine executable instructions for a physiological signal reconstruction system that cause a processor core to execute operations, the operations comprising:
extracting a raw physiological signal from a facial video sequence;
encoding the raw physiological signal into a latent representation of the raw physiological signal;
generating a noisy latent representation by applying forward diffusion to progressively add noise to the latent representation over a first set of iterations;
generating a denoised latent representation by applying reverse diffusion to progressively remove noise from the noisy latent representation over a second set of iterations; and
decoding the denoised latent representation to generate a reconstructed physiological signal.

2. The non-transitory machine-readable medium of claim 1, wherein the operations further comprise extracting one or more regions of interest from the latent representation.

3. The non-transitory machine-readable medium of claim 2, wherein progressively adding the noise to the latent representation comprises:
progressively adding a first noise to the one or more regions of interest; and
progressively adding a second noise to portions of the latent representation outside the one or more regions of interest, wherein the second noise has a greater magnitude than the first noise.

4. The non-transitory machine-readable medium of claim 2, wherein applying reverse diffusion comprises numerically integrating the noisy latent representation based on a Hamiltonian function over the second set of iterations, and wherein the Hamiltonian comprises an entropy regularization function associated with the one or more regions of interest.

5. The non-transitory machine-readable medium of claim 2, wherein the reverse diffusion employs a multi-component loss function that is a weighted sum of a set of loss components, wherein the set of loss components comprises a noise prediction loss that minimizes the discrepancy between predicted noise and the noise added during the forward diffusion.

6. The non-transitory machine-readable medium of claim 5, wherein the set of loss components comprises a region-guided refinement loss that applies additional constraints associated with the one or more regions of interest and a morphology of the reconstructed physiological signal.

7. The non-transitory machine-readable medium of claim 5, wherein the set of loss components comprises a fiducial point loss that penalizes misalignment of the one or more regions of interest.

8. The non-transitory machine-readable medium of claim 2, wherein the reconstructed physiological signal is an electrocardiogram (ECG) signal and the one or more regions of interest comprise at least one of a P-wave, a QRS complex, or a T-wave.

9. The non-transitory machine-readable medium of claim 2, wherein the reconstructed physiological signal is a photoplethysmography (PPG) signal and the one or more regions of interest comprise at least one of a systolic peak, a diastolic notch, or a diastolic peak.

10. The non-transitory machine-readable medium of claim 1, the operations further comprising:
extracting an additional raw physiological signal from the facial video sequence;
encoding the additional raw physiological signal into an additional latent representation of the additional raw physiological signal;
generating an additional noisy latent representation by applying additional forward diffusion to progressively add noise to the additional latent representation over a third set of iterations;
generating an additional denoised latent representation by applying additional reverse diffusion to progressively remove noise from the additional noisy latent representation over a fourth set of iterations; and
decoding the additional denoised latent representation to generate an additional reconstructed physiological signal,
wherein the reconstructed physiological signal is one of an electrocardiogram (ECG) signal or a photoplethysmography (PPG) signal and wherein the additional reconstructed physiological signal is the other of the ECG signal or the PPG signal.

11. The non-transitory machine-readable medium of claim 1, wherein the operations further comprise determining whether the reconstructed physiological signal is associated with a user based on a comparison between the reconstructed physiological signal and a ground truth physiological signal associated with the user.

12. The non-transitory machine-readable medium of claim 1, wherein the operations further comprise analyzing the reconstructed physiological signal to determine whether the reconstructed physiological signal is associated with a given health condition.

13. The non-transitory machine-readable medium of claim 1, wherein the operations further comprise analyzing the reconstructed physiological signal to determine at least one of a heart rate or an oxygen saturation associated with the reconstructed physiological signal.

14. The non-transitory machine-readable medium of claim 1, wherein extracting the raw physiological signal comprises extracting the raw physiological signal via a three-dimensional convolutional neural network (3DCNN).

15. A non-transitory machine-readable medium having machine executable instructions for training a physiological signal reconstruction system that cause a processor core to execute operations, the operations comprising:
accessing a training dataset comprising a set of training samples, wherein a training sample of the set of training samples comprises a facial video sequence and a ground truth physiological signal synchronized with the facial video;
extracting a set of raw physiological signals from facial video sequences of the set of training samples, wherein a raw physiological signal of the set of raw physiological signals is extracted from the facial video sequence via a three-dimensional convolutional neural network (3DCNN);
encoding the set of raw physiological signals into a set of latent representations, wherein a latent representation of the set of latent representations is generated by encoding the raw physiological signal via an encoder network;
generating a set of noisy latent representations from the set of latent representations, wherein a noisy latent representation of the set of noisy latent representations is generated via forward diffusion by progressively adding noise to the latent representation over a first set of iterations; and
training a reverse diffusion model to generate a set of denoised latent representations from the set of noisy latent representations, wherein a denoised latent representation of the set of denoised latent representations is generated by progressively removing noise from the noisy latent representation over a second set of iterations, and wherein the reverse diffusion model is trained based on the ground truth physiological signal and a reconstructed physiological signal generated by decoding the denoised latent representation.

16. The non-transitory machine readable medium of claim 15, wherein the raw physiological signal and the reconstructed physiological signal are both photoplethysmography (PPG) signals.

17. The non-transitory machine readable medium of claim 15, wherein the raw physiological signal and the reconstructed physiological signal are both electrocardiogram (ECG) signals.

18. The non-transitory machine readable medium of claim 15, wherein the training sample further comprises an additional ground truth physiological signal synchronized with the facial video, wherein the ground truth physiological signal is one of a photoplethysmography (PPG) signal or an electrocardiogram (ECG) signal, and wherein the additional ground truth physiological signal is the other of the PPG signal or the ECG signal.

19. The non-transitory machine readable medium of claim 15, wherein the set of training samples comprises at least one training sample for each Fitzpatrick skin type.

20. A method, comprising:
extracting a raw physiological signal from a facial video sequence;
encoding the raw physiological signal into a latent representation of the raw physiological signal;
generating a noisy latent representation by applying forward diffusion to progressively add noise to the latent representation over a first set of iterations;
generating a denoised latent representation by applying reverse diffusion to progressively remove noise from the noisy latent representation over a second set of iterations; and
decoding the denoised latent representation to generate a reconstructed physiological signal.
